(19) United States
(12) Patent Application Publication
(10) Pub. No.: US 2004/0001878 A1
(43) Pub. Date: Jan. 1, 2004

(54) Infused wound care dressings

(75) Inventors: Autry O.V. DeBusk, Knoxville, TN (US); Tim Alleman, Knoxville, TN (US)

Correspondence Address: CHAPMAN AND CUTLER, 111 WEST MONROE STREET, CHICAGO, IL, 60603, US

(73) Assignee: DeROYAL INDUSTRIES, INC., Powell, TN

(21) Appl. No.: 10/180,282

(22) Filed: Jun. 26, 2002

(51) Int. Cl. A61K 31/717; A61K 31/375; A61K 31/355; A61L 15/00

(52) U.S. Cl. 424/445; 514/60; 514/458; 514/474

(57) ABSTRACT

Healing of wounds is promoted by covering and contacting the wound with a dressing that has been infused with a suspension of a starch hydrolysate containing ascorbic acid and other components, such as collagen and alpha-tocopherol acetate. In suspension, in the infused dressing or carrier the combinations avoid the tendency of the components to separate thereby causing them to remain in suspension and to form a stable gel or dressing. Combining the starch hydrolysate and other components for delivery via the infused dressing allows for a more stable suspension for delivering the components to the wound. The infused dressing also permits the inclusion components that would be otherwise difficult to combine in a suspension. Wounds, in particular those occurring in the skin as second and third degree burns, stasis ulcers, trophic lesions, such as decubitus ulcers, severe cuts and abrasions which are commonly resistant to the natural healing process, may be treated with the infused dressing of the present invention.

### BACKGROUND OF THE INVENTION

1. Field of the Invention

This invention relates to infused dressings that provide an advanced healing environment for wounds, and, in particular, to any type of dressing that consists of a liquid phase before subsequently being processed into some type of film or semi-solid gel dressing. The process of wound healing is complex and good wound healing is characterized by rapid and complete regeneration of the damaged tissue. Considerable efforts have been expended in the study of wound dressings with the aims of finding which compounds and dressings are most effective in promoting wound healing. High macrophage activity is desirable, particularly during the early stages of healing, to kill bacteria and to remove cell debris and foreign matter. This activity is generally accompanied by inflammation. High fibroblast activity is desirable, particularly during the later stages of healing, to produce a high rate of regeneration.

2. Background of the Invention

Healing of wounds can be promoted by protecting and covering the wound surfaces with an infused dressing having a suspension of a starch hydrolysate containing ascorbic acid and other components. Combining the starch hydrolysate and other components for delivery via the infused dressing allows for a more stable suspension for delivering the components. The infused dressing also permits the inclusion components that would be otherwise difficult to combine in a suspension, such as silver nitrate and silver sulfur diozene. One such suspension of a starch hydrolysate containing ascorbic acid and other components is sold under the brand name Multidex and this formulation can be infused in a number of different classes of dressings which have the common characteristic of being sheet-type, wafer-type or film-type dressings that at some point in their preparation are in a liquid state. Three broad classes of these types of dressings are hydrocolloids, hydrogels, and polyacrylates.

A preferred generic hydrocolloid is made up of carboxy methyl cellulose and other components. A suitable hydrogel is primarily comprised of water (about 55%) and polyvinyl pyrrolidone. A typical polyacrylate consists of a combination of a polyacrylate and water. Typically, these types of dressings are prepared in a liquid phase and then are either treated or dried on a substrate in order to form the sheet, wafer or film dressing.

These types of dressings are initially prepared in a liquid phase that allows the addition of Multidex to the dressing. The target is to add about 5% to 35% (or a clinically effective amount) by weight of Multidex powder to the liquid phase to provide for either a Multidex containing solution or suspension that is subsequently prepared in the standard manner to result in the final Multidex containing dressing.

The process of wound healing consists of three phases during which the injured tissue is repaired, regenerated, and new tissue is reorganized into a scar. These three phases are classified as: a) an inflammation phase which begins from day 0 to 3 days, b) a cellular proliferation phase from 3 to 12 days, and c) a remodeling phase from 3 days to about 6 months. In all three phases, antioxidants play a vital role in the healing process.

In the inflammation phase, inflammatory cells, mostly neutrophils, enter the site of the wound followed by lymphocytes, monocytes, and later macrophages. The neutrophils that are stimulated begin to release proteases and reactive oxygen species into the surrounding medium with potential adverse effects on both the adjacent tissues and the invading microorganisms. The oxygen species known to be released by the neutrophils are superoxide (O₂⁻) through the action of a plasma membrane-bound NADPH oxidase, hydrogen peroxide (H₂O₂) formed by action of dismutation of O₂⁻, and HOCl produced by the action of myeloperoxidase with H₂O₂.

The proliferative phase consists of laying down new granulation tissue, and the formation of new blood vessels in the injured area. The fibroblasts, endothelial cells, and epithelial cells migrate in the wound site. These fibroblasts produce the collagen that is necessary for wound repair. Ascorbic acid is crucial in the formation of collagen. Several studies have demonstrated that ascorbic acid was capable of overcoming the reduced proliferative capacity of elderly dermal fibroblasts, as well as increasing collagen synthesis in elderly cells by similar degrees as in newborn cells even though the basal levels of collagen synthesis are age dependent. A decrease of ascorbic acid at the injury area will decrease the rate of wound healing.

In reepithelialization, epithelial cells migrate from the free edges of the tissue across the wound. This event is succeeded by the proliferation of epithelial cells at the periphery of the wound. Research has also shown that reepithelialization is enhanced by the presence of occlusive wound dressings which maintain a moisture barrier.

The final phase of wound healing, which is remodeling, is effected by both the replacement of granulation tissue with collagen and elastin fibers and the devascularization of the granulation tissue. Recent studies have shown that topical application of antioxidants, especially alpha-tocopherol, reduces scarring and normalizes blood coagulation during therapy.

As described in U.S. Pat. No. 4,778,679 which is incorporated by reference herein, a particularly effective healing treatment for wounds and skin defects such as burns, ulcers and lesions is the application of a medicinal dressing containing as an essential ingredient starch hydrolysate having Dextrose Equivalent of less than about 35. In such wound treatment the starch hydrolysate produces the formation of a film which is intimately adhered to the underlying granulation tissue and which is semi-permeable to gas and fluids and provides an ideal protective cover that will reduce fluid and plasma losses and invasion by pathogenic bacteria. In addition, it appears that the starch hydrolysate provides a topical or local hyperalimentation, that is local nutrition, providing a gradual release of glucose which is particularly effective in nutrition of tissue, both damaged and nascent, which have become relatively isolated from normal blood flow nutrition. The cessation of blood flow to such an ischemic lesion can be developed in a slow and gradual form such as in the case of decubitus ulcers and stasis ulcers, or may take place more acutely such as in thermo-radiation and chemical burns. In the absence of nutrition, the rate of fluid delivery of nutrients decreases bringing a progressive impairment in the viability of cells and tissues. This eventually leads to degeneration and death of the tissue and cells in a condition known as necrosis. Necrosis is generally accompanied by bacterial, fungal and/or viral contamination. As further pointed out in the aforementioned patent, treatment of exudative skin wounds with a starch hydrolysate dressing produces a greatly reduced bacteria count of an infected wound and inhibits infection of an uninfected wound. In addition, application of the starch hydrolysate to a wound or ulcer produces a film or semi-permeable membrane which allows edematous liquid to pass through while proteinaceous material is retained within the body, allowing reduction in the volume of exudate in relatively clean condition.

The present invention provides a sheet/wafer/film-type dressing infused with a combination of collagen type I, vitamins such as ascorbic acid (vitamin C) and alpha-tocopherol (vitamin E), and starch hydrolysate that is applied on wounds by covering the wound with the infused dressing to protect the wound and promote the formation and growth of healthy granulation tissue.

The infused dressing also permits the inclusion of components that would be otherwise difficult to combine in a suspension, such as silver nitrate and silver sulfur diozene. One such suspension of a starch hydrolysate containing ascorbic acid and other components is sold under the brand name Multidex and this formulation can be infused in a number of different classes of dressings which have the common characteristic of being sheet/wafer/film-type dressings that at some point in their preparation are in a liquid state. Three broad classes of these types of dressings are hydrocolloids, hydrogels, and polyacrylates.

A preferred generic hydrocolloid is made up of carboxy methyl cellulose and other components. A suitable hydrogel is primarily comprised of water (about 55%) and polyvinyl pyrrolidone. A typical polyacrylate consists of a combination of a polyacrylate and water. Typically, these types of dressings are prepared in a liquid phase and then are either treated or dried on a substrate in order to form the sheet/wafer/film dressing.

These types of dressings are initially prepared in a liquid phase that allows the addition of Multidex to the dressing. The target is to add about 5% to 35% (or a clinically effective amount) by weight of Multidex powder to the liquid phase to provide for either a Multidex containing solution or suspension that is subsequently prepared in the standard manner to result in the final Multidex containing dressing.

The repair process for even minor breaches or ruptures takes a period of time extending from hours and days to weeks; and in some instances, as in ulceration, the breach or rupture may persist for extended periods of time, i.e., months or even years. At all times, be it brief or extended, the potential for invasion by pathogenic organisms or foreign substances continues until new tissue has been generated to fully close the rupture or breach.

The healing process is brought about by complex biological mechanisms generally involving several groups of special cells and proteins. Leukocytes, such as neutrophils and macrophages, crown the wound site and digest foreign pathogens and debris. Such cells also send out chemical signals that marshal fibroblasts in the wound vicinity and ultimately generate connective structures, principally, collagen, which make up a major portion of the new tissues. Endothelial cells generate new blood capillaries that grow into the reconstructed tissue areas where their presence is necessary to supply nutrients to the newly growing tissue cells and remove catabolic products. As the new capillaries grow, the cells on the margin of the wound simultaneously multiply and grow inwardly. The fibrous tissue arising from this cell growth eventually fills the wound cavity with a network of interlacing threads of collagen which in due time, arrange themselves in firm bands and form the permanent new tissue.

The surface of the wound is subsequently covered by processes of enlargement, flattening, and multiplication of the epithelial cells at the wounds' edge. These epithelial cells spread as sheets into the wound, beneath the scab. Eventually the proliferating epithelial cell sheets emanating from the wound sides coalesce to cover and close the wound on the outer surface.

Until such time as at least superficial healing has occurred, or healing is impaired, the individual remains at risk from continued or new infection. Hence, there is a time versus rate related risk factor involved in all wound situations. The quicker the wound can heal, the sooner the risk is removed. Therefore, any procedure that can influence the rate of wound healing, or even favorably influence the healing of intractable wounds, would be of paramount value.

In accordance with this invention, improvements in the starch hydrolysate treatment of wounds have been developed to provide dressing for wound treatment, effective to promote the healing process, as well as beneficial compounding of the starch hydrolysate material.

### SUMMARY OF THE INVENTION

This present invention relates to compositions, methods and procedures that improve the ability of wounds to heal and/or increase the rate at which wounds heal.

More specifically, the present invention provides an infused dressing which, when applied to wounds, greatly enhance and promote the normal healing processes. The dressing is infused with compositions comprising a suspension of a mixture of the fibrous protein, collagen type I; ascorbic acid; alpha-tocopherol acetate; and a starch hydrolysate.

In the process of the invention, the collagen type I/ascorbic acid/alpha-tocopherol/starch hydrolysate suspension is infused into a carrier or dressing which is then applied to cover the wound and maintained in contact therewith for an extended period, i.e., during the entire healing process, or until closure of the wound by new tissue has taken place.

It is therefore an object of the invention to provide an infused dressing that promotes the tissue healing process.

It is further another object of the invention to provide a dressing infused with a composition of collagen type I/ascorbic acid/alpha-tocopherol/starch hydrolysate.

Another objective of this invention is to provide collagen type I/ascorbic acid/alpha-tocopherol/starch hydrolysate compositions as an article that promotes wound healing; reduces scarring and may yet normalize blood coagulation during therapy.

Another object of the invention is to provide a carrier or dressing utilizing collagens, hydrophilic foams, hydrocolloids, amorphous hydrogels, hydrogel sheets, polyacrylates or similar porous materials.

Another object of the invention is to provide a starch hydrolysate containing ascorbic acid and other components that is sold under the brand name Multidex. This formulation can be infused in a number of different classes of dressings which have the common characteristic of being sheet/wafer/film-type dressings that at some point in their preparation are in a liquid state.

Another object of the invention is to provide a starch hydrolysate containing ascorbic acid and preferred generic hydrocolloid made up of carboxy methyl cellulose and other components. A suitable hydrogel is primarily comprised of water (about 55%) and polyvinyl pyrrolidone. A typical polyacrylate consists of a combination of a polyacrylate and water. Typically, these types of dressings are prepared in a liquid phase and then are either treated or dried on a substrate in order to form the film dressing to contain 5% to 35% (or a clinically effective amount) by weight of Multidex powder to the liquid phase.

### DESCRIPTION OF THE PREFERRED EMBODIMENT

The present invention relates to dressings, compositions and methods that improve the ability of skin wounds, e.g., second and third degree burns, stasis ulcers, trophic lesions such as decubitus ulcers, severe cuts and abrasions to heal and/or increase the rate at which wounds heal. Healing of wounds is promoted by contacting the wound surfaces with an infused dressing having a suspension of a starch hydrolysate containing ascorbic acid, collagen type I and alpha-tocopherol acetate and other components. These combinations are present in suspension in an infused dressing or carrier to avoid the tendency of the components to remain in suspension in order to form a stable gel or powder. Combining the starch hydrolysate and other components for delivery via the infused dressing allows for a more stable suspension for delivering the components. The infused dressing also permits the inclusion of other components, such as silver nitrate or silver sulfur diozene, that would be otherwise difficult to combine in a suspension. Wounds, in particular those occurring in the skin as second and third degree burns, stasis ulcers, trophic lesions, such as decubitus ulcers, severe cuts and abrasions which are commonly resistant to the natural healing process, may be treated with the infused dressing of the present invention.

A wound may be defined as an incision or a trauma to any of the tissue of the body. The wound healing is a complex continuous process and has often been divided into several overlapping stages.

Hemostasis or stoppage of bleeding in the first step of any wound healing process. Blood platelets and soluble clotting factors are the major intravascular hemostatic factors. Collagen is a very efficient hemostatic agent because platelets adhere to collagen, swell and release substances, which initiate hemostasis. Collagen can provide the positive and negative active polar sites as well as a molecule of sufficient size for platelet aggregation.

Properly configured bovine collagen, which preserves collagen's molecular structure, is dramatically different from products, which utilize a perverted form of the collagen molecule. The difference is analogous to the difference between particleboard and wood. Both are made of the same raw materials; however, with particleboard the integrity of the raw material is compromised and then reconstituted using a foreign substance. This results in dramatically different composition and effectiveness of the product. Changing the molecular structure of the product results in an even more profound difference. A molecule which does not maintain its basic properties essentially becomes a different molecule.

Skin wounds are characterized by openings or gaps in the skin tissue. As the healing process progresses, these open wounds are gradually filled in by new cells that appear across the surface of the wound, so that by the time the healing process is complete, new skin tissue covers the former open area of the wound. Such cells are termed granulation cells and the healing mechanism is a granulation cell formation process. These granulation cells are, however, very fragile and rupture easily. Heretofore, conventional dry gauze dressings have been used widely on such burns or exudative lesions. When dry gauze is removed, as for example when it is changed, the cells rupture; thus temporarily arresting the healing process. A dressing for burns and exudative lesions, therefore, is preferably infused with a combination of healing agents and should be capable of removal without disturbing the growth of the very fragile granulation cells.

Generally, after a would, the body rushes edema fluids to the area of these skin wounds and such wounds usually exude this edema liquid. Consequently, vital body fluids are lost in the exudate. If the loss of such fluids is great enough, shock ensues. Heretofore, the conventional technique to prevent this loss of vital body fluids has been to attempt to seal off the exuding wound. This has been accomplished, for example, by applying to the wound a layer of petrolatum or other water immiscible gelatinous hydrocarbon material. However, it has been found that the tissue under such a layer of petrolatum is often excessively soft and wet. This softened tissue causes difficulty in both autograft and homograft skin transplants. It also provides an environment that is conducive to the growth of secondary infections. The wound, therefore, must be cleansed constantly. But cleansing necessitates removal and replacement of the dressing and, as described above, there is great danger of rupturing the very fragile granulation cells during this removal and replacement process.

An improved method for treatment of the aforementioned skin wounds, should therefore, employ an infused dressing that is permeable and flexible and inhibits the start of secondary infections by reducing the bacteria count around the treated wound. In the treatment of second and third degree burns, the dressing employed should be similar to a skin autograft in that it affords a natural protective covering which promotes healing, and yet it should, like a homograft, be easily sloughed off by the body when the healing process is completed.

The present invention provides a new and novel method of treating skin wound that comprises the use of a sterile, infused dressing which is applied to the wound. The dressing is infused with at least, purified starch hydrolysate material having a DE of less than about 35. DE is an abbreviation for “Dextrose Equivalent,” which is an expression in the art for describing the total reducing sugar content of a material calculated as dextrose, and expressed as percent, dry basis. A low DE starch hydrolysate product is one having a DE of less than about 35. As heretofore has been mentioned, in practicing the invention we use a sterile, purified starch hydrolysate material having a DE of less than about 35, however, the preferred sterile, purified starch hydrolysate materials have a DE of between about 5 and about 25.

The infused dressing is porous and will create a semi-permeable membrane which allows edema liquids to pass through it while proteinaceous materials are retained within the body. The exudate is clean and relatively free of proteinaceous materials. It, therefore, does not support biological oxidation to the same extent as exude containing proteinaceous fluids is minimized while at the same time excessive build up of edema liquids is also minimized.

To this end, the sterile, purified starch hydrolysate particulate material having a DE less than about 35 may be admixed with any of the antibacterial agents known to the art to be effective in the prevention, and/or treatment of secondary infections, e.g., iodine, penicillin, nitrofuranes and the sulfa drugs such as silver sulfadiazine. In addition, proteolytic enzymes known by the art to be effective in promoting healing may also be admixed with this particulate material. Furthermore, collagen type I, nutritive agents, such as amino acids, cystine and cysteine, vitamins such as ascorbic acid (vitamin C) and alpha-tocopherol (vitamin E), and particulate starch hydrolysate may also be admixed prior to infusion to promote the formation and growth of healthy granulation tissue.

To promote the formation of this protective film, the infused dressing should not be applied too tightly, but should permit the wound to “breathe.” It has been found that application of this material serves also to reduce the pain that is usually associated with burns, ulcers, and the like. These dressings also have the aforementioned properties of being flexible, semi-permeable, and antiseptic to the bacteria of the wound if admixed with antibacterial agents.

The present invention provides a sheet/wafer/film-type dressing infused with a combination of collagen type I, vitamins such as ascorbic acid (vitamin C) and alpha-tocopherol (vitamin E), and particulate starch hydrolysate that are applied on wounds by covering the wound with the infused dressing to protect the wound and promote the formation and growth of healthy granulation tissue.

The infused dressing also permits the inclusion components that would be otherwise difficult to combine in a suspension, such as silver nitrate and silver sulfur diozene. One such suspension of a starch hydrolysate containing ascorbic acid and other components is sold under the brand name Multidex and this formulation can be infused in a number of different classes of dressings which have the common characteristic of being film-type dressings that at some point in their preparation are in a liquid state. Three broad classes of these types of dressings are hydrocolloids, hydrogels, and polyacrylates.

### HYDROCOLLOID DRESSINGS

Hydrocolloids consist predominantly of a suspension of gel-forming polymer, gums and adhesive on a film or foam backing. Hydrocolloid dressings promote a moist wound environment, aid in autolytic debridement and have no particulate or toxic components. They are waterproof, bacteriaproof and conformable. The entire wound contact surface is adhesive until the dressing comes into contact with wound exudate. The exudate is absorbed by the dressing, which converts into a soft gel. The area of the dressing in direct contact with the wound itself loses its adhesiveness and thus will not damage the wound surface on removal of the dressing.

Hydrocolloid dressings are available in thick (regular) and thin version in a variety of shapes and sizes, as well as a paste and a powder for wetter wounds or to fill cavities. Hydrocolloid dressings are also available in combination with an alginate to increase the fluid handling capability of the dressing. Hydrocolloids can be used on lightly to moderately exudating wounds such as leg ulcers, pressure wounds, burns, donor sites etc. Thin hydrocolloids can also be used over suture lines and at IV sites. Hydrocolloids are useful in the prevention of pressure areas. As the dressing material itself may be adhesive, the edges of the dressing are prone to rolling and can leave residues of adhesive on clothing and bedding. This problem can be circumvented by placing a border of tape around the edges of the dressing or using the dressings with bevelled edges.

A preferred generic hydrocolloid is made up of carboxy methyl cellulose and other components. A suitable hydrogel is primarily comprised of water (about 55%) and polyvinyl pyrrolidone. A typical polyacrylate consists of a combination of a polyacrylate and water. Typically, these types of dressings are prepared in a liquid phase and then are either treated or dried on a substrate in order to form the film dressing.

These hydrocolloid dressings will create a moist environment that facilitates wound healing. The dressing material (containing absorbent granules embedded in an adhering matrix) interacts with wound exudate by forming a soft gel. Due to its matrix formulation, most of the gel is removed together with the dressing with little or no damage to the newly formed tissue. Some hydrocolloid dressings made in accordance with this invention may have a wound contact layer of hydrocolloids and a top layer made of a semi-permeable low friction polyurethane film.

Hydrocolloid dressings may also contain an adhesive layer or edge that helps protect skin against maceration and excessive moisture. A low profile design will permit dressings to easily conform to difficult body contours, while the smooth outer surface helps protect the skin from shear and friction. Bordered dressing preferably have a tapered edge which is particularly good against external bacterial contamination maintains moist wound environment.

Hydrocolloids are a type of dressing containing gel-forming agents, such as sodium carboxymethylcellulose (NaCMC) and gelatin. In many products, these are combined with elastomers and adhesives and applied to a carrier—usually polyurethane foam or film, to form an absorbent, self adhesive, waterproof wafer.

In the presence of wound exudate, hydrocolloids absorb liquid and form a gel, the properties of which are determined by the nature of the formulation. Some dressings form a cohesive gel, which is largely contained within the adhesive matrix; others form more mobile, less viscous gels which are not retained within the dressing structure.

In the intact state most hydrocolloids are impermeable to water vapor, but as the gelling process takes place, the dressing becomes progressively more permeable. The loss of water through the dressing in this way enhances the ability of the product to cope with exudate production. Hydrocolloids are easy to use, require changing only every 3-5 days, and do not cause trauma on removal. This makes them useful for clean, granulating, superficial wounds, with low to medium exudate. Hydrocolloids provide effective occlusion; with dry wounds, they can have a softening effect, and they have been used to prevent the spread of MRSA (by providing a physical occlusive barrier).

Hydrocolloid wound dressings have been in use for some 20 years, but they have not been combined as described in this invention to provide the described beneficial results. The ability of hydrocolloids to absorb fluids varies over time and between products so that some may not be suitable for medium to high exuding wounds.

Over recent years, many new dressings have appeared on the market, but few new dressing types. The continuing success of hydrocolloids depends largely on their effectiveness as occlusive dressings. Any new dressing has to match or better their performance and/or compete on price. Currently, polyurethane foam dressings are being promoted as an alternative to hydrocolloids and could be infused as contemplated herein.

These types of dressings are initially prepared in a liquid phase that allows the addition of Multidex to the dressing. The target is to add about 5% to 35% (or a clinically effective amount) by weight of Multidex powder to the liquid phase to provide for either a Multidex containing solution or suspension that is subsequently prepared in the standard manner to result in the final Multidex containing dressing.

The preferred Multidex formulation is currently 98% maltodextrin, 1% ascorbic acid and 1% fructose (again by weight). Obviously, any of the other alternative embodiments of Multidex could be used as disclosed in the following patents, assigned to the assignee of the present invention: U.S. Pat. No. 6,187,743, U.S. Pat. No. 6,046,160, U.S. Pat. No. 6,046,178, U.S. Pat. No. 5,177,065, U.S. Pat. No. 4,889,844, U.S. Pat. No. 4,778,679, U.S. Pat. No. 4,414,202.

Starch is a polymer or anhydro D-glucose unit. Hydrolysis of starch produces a mixture of polymers of various molecular weights ranging from 200 glucose units or more down to maltose (2 glucose units) and D-glucose itself. Because of their nature the accepted way to describe the polymers formed by hydrolysis of starch is by their DE value, which is an expression of the average extent of hydrolysis.

### HYDROGELS

Hydrogels are water swollen networks of water-loving polymers and synthetic hydrogel applications have began to grow at a fast pace. Hydrogels are water-swollen networks of hydrophilic homopolymers or copolymers. These networks may be formed by various techniques, however the most common synthetic route is the free radical polymerization of vinyl monomers in the presence of a difunctional crosslinking agent and a swelling agent. The resulting polymer is interesting in that it exhibits both liquid-like and solid like properties. The liquid-like properties result from the fact that the major constituent (>80%) is water. However, the polymer also exhibits solid-like properties due to the network formed by the crosslinking reaction. These solid-like properties take the form of a shear modulus which is evident upon deformation.

If an ionic or hydrophobic monomer is incorporated into the hydrogel network, a responsive polymer is often created. This responsiveness takes the form of a volume phase transition, which is characterized by a sudden change in the degree of swelling upon a small change in environmental conditions. This behavior follows the trends seen in linear polymer systems showing response to environmental pH, salt concentrations, and temperature. For example it is known that poly(isopropylacrylamide) contains a lower critical solution temperature (LCST) at ˜34° C. Likewise isopropylacrylamide hydrogels undergo a discrete collapse of the polymer network ˜32° C. Discrete changes in swelling behavior may also be seen in hydrogels incorporating a monomer containing a carboxylic acid moiety. Therefore, with changes in pH, the hydrogel's charge density will change and thus, the swelling behavior of the gel. By changing the amount of water associated with the network, one is effectively changing the hydrophilic/hydrophobic balance and therefore, one may utilize these systems to reversibly interact with hydrophobic materials.

A hydrogel is a bio-material component, which is filled with water up to a point of equilibrium. This ability to absorb large quantities of water allows a hydrogel to resemble living material. Most of our tissues are made up of protein hydrogels, lipid hydrogels and sugar hydrogels. A hydrogel absorbs all the water it can find up to its maximum capacity. It acts like water in maintaining its spatial geometiy and remains elastic like rubber.

### HYDROPHILIC FOAMS

Another form of carrier or dressing suitable for infusion includes a class of products known as hydrophilic foams. Hydrophilic foams are available in sheets and other shapes of formed solutions of polymers, most commonly polyurethane. The small open cells are capable of attracting and holding fluids and can be used as either primary or secondary dressings. Hydrophilic foams are commercially available with or without adhesive borders. Sometimes referred to as medical foams, these products are extremely soft and absorbent, while being highly breathable. They are elastic and non-toxic and conform to complex shapes while resisting the effects of gasses. They have the ability to absorb up to 20 times their own weight, and have been found to be particularly effective for incontinent pads and foot pad inserts. They are suitable for long-term wear and tolerant to gamma sterilization and, thus, are suitable for infusion of the wound care composition of the present invention.

These broad classes or types of dressings are hydrocolloids, hydrogels, hyrdophilic foams and polyacrylates, but the invention contemplates the use of any type of dressing that consists of a porous membrane that may begin in a gel or liquid phase before subsequently being processed into some type of film or semi-solid gel dressing. Some of these dressings are initially prepared in a liquid phase where Multidex is added to the dressing to about 5% to 35% (or a clinically effective amount) by weight of Multidex powder to the liquid phase to provide for either a Multidex containing solution or suspension that is subsequently prepared in the standard manner to result in the final Multidex containing dressing.

Low DE products suitable for use in the present invention, can be made by subjecting the gelatinized starch to the hydrolytic action of an acid, or an enzyme and/or successive treatments with such agents. The hydrolysate so formed is then purified by conventional means such as by subjecting it to filtration, centrifugation, decantation or the like to separate and remove any water insoluble materials remaining after hydrolysis. This material, dissolved in water to the extent of 10 grams per 100 ml will contain less than 0.1 percent insoluble materials as determined by filtration and drying the residue to constant weight under vacuum at 100° C. If desired the hydrolysate material may be subjected to further purification steps known to the art such as carbon or clay treatment, dialysis, electro-dialysis, osmosis, ion exclusion, ion exchange and the like. The starch hydrolysate material employed in practicing the invention may by prepared from starch by a number of specific methods.

Suitable starch hydrolysate materials may also be made via a number of other routes. For example, a mixture of starch and water having a solid content less than 50 percent may be first subjected to the hydrolytic action of a bacterial alpha-amylase. After an initial thinning by the enzyme, the resulting partial hydrolysate is heated to a temperature sufficient to solubilize any unsolubilized starch. Since this temperature also tends to inactivate the enzyme, it is then necessary to subject the solubilized partial hydrolysate to a second hydrolysis by treatment with more bacterial alpha-amylase to obtain the final starch hydrolysate before infusing into the dressing.

Any starch or starch like material may be used to prepare the starch hydrolysate material used in the invention. Suitable materials include cereal and tuber-starches, such as corn, wheat, potato, tapioca, rice, sago and grain sorghum, waxy starches may also be used. Hydrolysis may be carried out by enzymes, acids or in combinations of the two.

The hydrocolloids, hydrogels, and polyacrylates dressings used in practicing the invention should, of course, be sterile. Sterilization may be accomplished by any of the known sterilization procedures.

The use of free-radical scavengers as anti-inflammatory drugs is discussed in the Handbook of Inflammation, Volume 5: The Pharmacology of Inflammation pages 255-281 (Elsevier Science Publishers BV, 1985). Superoxide anion serve an indispensable purpose in the killing of phagocytosed microorganisms. Superoxide anion and reaction products such as hydroxyl radical are said to be deleterious in the extracelluar environment, possibly leading to self-maintenance of the inflammatory reaction and to tissue damage. The authors report the successful treatment of a variety of inflammatory conditions by injection of the enzyme superoxide dismutase (SOD), which catalyses the reaction:

20₂⁻+2H⁺→O₂+H₂O₂

The authors point out a number of difficulties in the concept of scavenging free radicals by drugs rather than by enzymes. In particular, the reaction of free radical and scavenger generates a new free radical. If this is as reactive as the original radical, no benefit has been obtained. If it is less reactive, it may migrate away from the wound site and cause damage in a previously healthy area. The authors conclude that scavenging of highly reactive radicals in vivo is neither feasible nor desirable.

Chemical groups that are precursors for persistent free radicals contain no unpaired electrons. They are capable of reacting with active free radicals to produce persistent free radicals. Polymers that carry such precursor groups are generally preferred to those that initially carry persistent free radicals, for a number of reasons. There are a wide variety of such groups, and they are generally chemically stable and easy to prepare. Suitably chosen groups can engage in more than one free radical reaction, whereas persistent free radicals can in general engage in no more than one. In such a case, the precursor group reacts with an active free radical to form a persistent free radical, which subsequently quenches a second active free radical. Such precursor groups may engage in more than one free radical reaction in a variety of ways, depending on the chemistry of the substances involved.

U.S. Pat. No. 5,667,501 describes compositions which a polymer p¹ scavenges an active-free radical R. to form a persistent free radical (P¹R), which subsequently quenches another active free radical R. by addition.

It was unexpected to find that polymers applied as wound dressings and remote from the wound on a molecular scale should be able to affect the wound healing process apparently through their ability to react with free radicals from the site of biological activity in the wound.

Low concentrations of hydrogen peroxide (around 10⁸-10⁶ M) have been shown to stimulate fibroblast proliferation. This is particularly desirable during the later stages of wound healing. Polymers that carry groups that are precursors of free radicals are often capable of reacting with molecular oxygen in a physiological environment to generate hydrogen peroxide. The process is catalyzed by iron ions present at physiological concentrations.

Vitamin C rescues vitamin E radical and regenerates vitamin E not only in homogeneous solution but also in liposomal membrane system. The efficiency or relative importance of vitamin C with vitamin E radical interaction is smaller in liposomal membrane system than in homogeneous solution. This is because vitamin C suppresses the consumption of vitamin E almost completely in homogeneous solution, whereas vitamin E is consumed appreciably even in the presence of vitamin C during the oxidation of liposomal membranes initiated with lipid-soluble radical initiator.

The lipid peroxyl radicals in the membrane must be scavenged exclusively by vitamin E. The vitamin E radical formed may undergo several competing reactions. It may scavenge another peroxyl radical to give stable product, react with another vitamin E radical to give a dimer, or interact with vitamin C to regenerate vitamin E. The lower the peroxyl radical concentration, the higher is the efficiency of vitamin E regeneration. Apparently, this efficiency depends also on the accessibility of vitamin C to the vitamin E radical in the membrane.

The close proximity of the chromanol head group to the membrane surface is consistent with the synergistic antioxidant behavior of vitamins C and E observed in peroxidations of artificial phospholipid membranes using lipid-soluble, thermal azo initiators. Although the two vitamins are completely sequestered and separated in their respective liquid and aqueous phases, a very significant extension of inhibition of peroxidation is obtained when both are present. Vitamin C by itself is a good antioxidant when peroxyl radicals are generated in the aqueous phase, but it is very much less effective when radicals are generated within a membrane. Presumably, vitamin C can not penetrate the membrane sufficiently to interact with a peroxyl radical present there. The most likely explanation of the synergy between vitamins C and E is that vitamin C is able to reduce the tocopheroxyl radical back to alpha-tocopherol. The chemical feasibility of this regeneration mechanism has been amply demonstrated in homogenous media. A decrease of vitamin C at the site of injury will decrease the rate of wound healing.

Vitamins C and E function as water-soluble and lipid-soluble chain-breaking antioxidants, respectively, and protect lipids, proteins, and membranes from oxidative damage. Vitamin C scavenges oxygen radicals in the aqueous phase, whereas vitamin E scavenges oxygen radicals within the membranes. Vitamin C regenerates vitamin E by reducing vitamin E radicals formed when vitamin E scavenges the oxygen radicals. This interaction between vitamin C and vitamin E radicals can take place not only in homogeneous solutions but also in liposomal membrane systems where vitamins C and E reside separately outside and within the membranes respectively, and vitamin C can act as a synergist.

The effect of vitamin E on wound healing is complex. It has been determined that vitamin E should be present in the aqueous dispersion at a concentration of about 8-10 μg/ml. The relative concentration of the vitamin E component must be maintained within fairly well defined limits. If the concentration of vitamin E is too high, there is inhibitory effects or inflammatory reactions. The preferred concentration of vitamin E in this invention is 10 μg/ml.

Another function of these antioxidants, vitamins C and E is their chemotactic factors. Studies with patients in serious trauma indicate that the observed neutrophil locomotory dysfunction is partly due to auto-oxidation as shown by evidence of pre-activation of diminished reducing capacity and low serum and cellular levels of vitamin C and vitamin E.

In this invention, a medicinal dressing is infused with a suspension of a starch hydrolysate containing ascorbic acid, collagen type I and alpha-tocopherol acetate and other components. In particular the starch hydrolysate used is commercially available and sold under the trade name Multidex®. These combinations are in suspension in the infused dressing or carrier to avoid the tendency of the components to separate thereby causing them to remain in suspension and to form a stable gel or powder. Combining the starch hydrolysate and other components for delivery via the infused dressing allows for a more stable suspension for delivering the components. The infused dressing also permits the inclusion components that would be otherwise difficult to combine in a suspension. Wounds, in particular those occurring in the skin as second and third degree burns, stasis ulcers, trophic lesions, such as decubitus ulcers, severe cuts and abrasions which are commonly resistant to the natural healing process, may be treated with the infused dressing of the present invention.

The commercially available Multidex® combinations—particularly those including collagen and Vitamin E, may not remain in suspension in order to form a stable gel or powder. Combining one of these Multidex® formulations with the components in a “suspension absorbing” sheet carrier or gel dressing allows for a more stable suspension for delivering the components to the wound. Also, as described above, the carrier or dressing allows the possibility to combine components that would not otherwise be combinable such as Multidex® and silver nitrate and/or silver sulfur diozene.

The hydrocolloids, hydrogels, and polyacrylates dressing could be provided in sheet form or in a more aqueous gel form as described below. The dressings or carriers can be formed of many different materials, but it has been found that certain types of materials are particularly effective in absorbing and holding the various combinations in suspension for delivery to the wound site. Additional dressing types are described below.

Collagens in the form of pads, sheets, particles, powders, pastes, or gels may be derived from bovine, porcine, or avian sources. It has been found that collagen plays a significant role in all phases of wound healing. Collagen lays out a matrix upon which skin grows and maintains its integrity. It is analogous to the wrought iron or steel infrastructure that provides the structural integrity to a high rise building or highway. When there is damage, the first step is to replace the infrastructure, since the finishing building materials cannot be applied until the infrastructure is in place.

Mammalian collagen accelerates the healing by increasing the concentration of cellular and non-cellular elements, including fibroblasts and growth factors. Collagen has many advantageous properties when put in contact with a wound bed. These properties include homeostatic effect, interaction with platelets, interaction with fibronectin, increase in fluid exude, increase in cellular component, increase in growth factors and support for fibroblastic and, eventually, epidermal proliferation.

Collagen and derivatives of collagen (gelatin) have been widely used in medical, pharmaceutical and consumer products for approximately 40 years. The supply of collagen is both abundant and inexpensive. However, most formulations of this material are not highly purified, and have the potential to cause an inflammatory reaction in some collagen product users. In addition, some concerns have been raised over the last years about the potential for contamination of bovine collagen with the fatal mad cow disease and its human variant, Creutzfeldt-Jakob Disease. Human salines have been used to naturally produce significant amounts of collagen, but such processes have yet to reach commercialization. Companies have also extracted human collagen from placenta and cadaver tissue; however, validation for the absence of disease causing organisms, consumer acceptance, and the availability of material raise serious limits for such technologies.

The hydrocolloids are available as wafers, powders, or pastes composed of materials such as gelatin, pectin, and carboxymethylcellulose Hydrocolloids vary in absorption capacity depending upon the thickness of the composition. Wafers are typically self-adhering and are also available with our without adhesive borders. Powder and paste type hydrocolloids would require a secondary dressing. Carbohydrate based flexible wafers may contain hydroactive particles, and do not adhere to a wound surface. They are generally impermeable to bacteria and are waterproof, thereby keeping a wound in a moist environment.

Another class of the dressings described above are hydrogels formulated of water, polymers, and other ingredients with no particular shape, and are designed to donate moisture to a dry wound to maintain a moist healing environment. The high moisture contact of amorphous hydrogels serve to re-hydrate wound tissue and typically require an outer or secondary dressing. Hydrogels are also available in sheet form, which are three-dimensional networks of cross-link hydrophilic polymers that are insoluble in water and interact with aqueous solutions by swelling. They are highly conformable and permeable, and can absorb various amounts of drainage from a wound depending upon their composition. These sheets are available with our without borders. Hydrogels can absorb a substantial amount of wound care composition for delivery to a wound, while also absorbing or subtracting moisture from the wound environment. They may be used on both superficial and deep wounds, and they have a high ability to absorb the wound care composition and moisture from a wound.

Multidex® formulations can include ascorbic acid in admixture or application along with the starch hydrolysate material to promote the formation and growth of healthy granulation tissue. Ascorbate salts such as those of sodium, potassium and calcium can also be employed, though ascorbic acid is the preferred component for blending with the starch hydrolysate powder at a level in the range of approximately 0.5-20 weight percent of the blended composition. Preferably about 5-7.5 weight percent corresponding to a weight ratio of approximately 20 parts starch hydrolysate to one part ascorbic acid or ascorbate salt. While ascorbic acid appears somewhat more effective than the ascorbate salts and is less readily oxidized mixtures of ascorbic acid and ascorbate salt can also be employed in order to reduce acidity.

Generally, in the preparation of wounds for treatment with starch hydrolysate dressing, the patient selected for study is carefully examined, test areas photographed, and when possible the volume of the lesion measured. Biopsy, planimetric, bacterial culture, and sensitivity studies are made and thereafter the ulcer or wound is carefully, surgically debrided with all necrotic tissue removed mechanically. Enzymatic debridment can also be carried out when necessary, usually employing proteolytic enzymes such as Travase, Biozyme, collagenase, and Elase.

Broadly, the treatment procedure in accordance with this invention begins with irrigation of the wound, typically employing a syringe for pressure irrigation. Thereafter, the wound is covered with a medical dressing infused with a Multidex® formulation preferably containing both ascorbic acid and Vitamin E. A roller bandage or other secondary dressing may then be applied, if needed.

Another type of dressings suitable for infusion is collagen alone or combined with one of those described above. As wound healing has several distinct phases: inflammation: platelet aggregation, cell recruitment; granulation tissue formation: neovascularization; and extracellular matrix deposition: wound contraction. Components of the extracellular matrix, including collagen, are involved in every stage of wound healing. The first event, immediately following injury, is blood vessel disruption leading to extraversion of blood constituents, followed by platelet aggregation and blood coagulation. Collagen has a key role in these processes. Exposed collagen in the wound function to promote platelet aggregation following vascular injury.

Collagen is frequently described as a stable, relatively inert component of the extracellular matrix. While this statement may be true of the collagen deposited and cross-linked into an extracellular matrix, the primary role of which is to provide an extracelluar framework or scaffold to support cells; collagen has been increasingly considered to be dynamic proteins involved in many cellular and developmental processes. Distinct roles have been elucidated for collagen during morphogenesis and development, platelet adhesion and aggregation, cell attachment, cell migration, angiogenesis and filtration in basement membranes. Contemporary techniques to assess collagen turnover have also indicated that collagen metabolism is much more rapid than once considered.

Collagen is a proteinaceous material comprising the major fibrous element of the mammalian skin, bone, tendon, cartilage, blood vessels, and teeth. Its biological purpose is to hold cells together in discrete units; and secondarily it has a directive role in developing tissues. The collagen proteins are distinctive in their physical characteristics in that they form insoluble fibers possessing high tensile strength. It is the fibrous nature of the collagen that serves to hold the various body structures and components together.

While the basic molecular structure of collagen may be modified to meet the needs of particular tissues, all collagen are organized into a common structure consisting of three polypeptide chains that form a triple stranded helix. The triple stranded helical units, in turn, are formed into a quarter-staggered array of linearly aligned bundles that make up collagen fibers. The collagen fibers are stabilized by covalent cross-links.

It has been shown that purified collagen can be utilized medically in reconstruction and cosmetic surgery for the replacement of bony structures or gaps in bony structures, and for filling out tissues where wrinkles have forms. In such usage, collagen is secured from mammalian sources, e.g., calves, whereby extraneous proteinaceous material is removed by various dissolution, precipitation and filtration techniques to leave a pure collagenous product. Unfortunately this pure natural collagen may induce antigenic response in the host subject. Such antigenic response in the host may be generated by the end portions of the collagen fibrils that are not helically bound. Fortunately these end portions of collagen can be cleaved therefrom by treatment with a proteolytic enzyme, e.g. pepsin. After digestion with pepsin, the cleaved peptide ends are discarded and only the central collagen bundles (tropocollagen) remain. These central collagen bundles have greatly reduced antigenicity and they can be used for the purposes noted above without undue antigenic side effects.

Although reduced antigenic collagen is preferred, non-cleaved collagen that has been isolated from animal sources may also be used. It is only necessary that the collagen be prepared in a sterile condition in an aqueous suspension. Some inclusion of materials commonly associated with the collagen, e.g., polysaccharides, can be tolerated and do not interfere with the benefits of the wound healing compositions. Other forms of processed collagen are also useful in the compositions.

Another advantage of collagen is that when collagen is heated it can be denatured, and solubilized for easy application as a gelatin-like solution. When cooled, the collagen is partially renatured, resulting in a gel formation with excellent tensile strength. Heated collagen, therefore, is an ideal protein component in the present tissue bonding or sealing composition. Through heating, collagen can be solubilized and easily injected or applied, and by cooling it can be turned into a gel which provides both tensile strength and flexibility to the bond. Collagen can also be rendered in a sterile form. Moreover, collagen is more stable than its fibrin counterpart, both on the shelf and in vitro and collagen does not expose the recipient to the risk of contacting infection as does fibrin glue.

Collagen type I can be found in most tissues and organs, but is most plentiful in dermis, tendon, and bone. Type I collagen is a 300 ηm-long heterotrimer composed of two alpha (α₁) (I) chains and one α₂(I) chain. Collagen-binding integrin receptors are α₁β₁, α₂β1, and α₃β₁.

The collagen should be present in the aqueous dispersion at an optimal concentration of about 0.2-2.0 ug/cm². Considerable variation above or below the noted concentrations is permissible so long as undue inflammation does not occur. It is desirable to hold the concentrations close to the stated amounts to produce optimum results, and to avoid inhibitory effects or inflammatory reactions. The preferred concentration for collagen is 10 ug/cm².

U.S. Pat. No. 4,837,024 describes compositions which enhance and promote the wound healing process and which comprise suspensions of the fibrous protein, collagen, and of a polysaccharide, namely a glycosaminoglycan. The glycosaminoglycan is one which exhibits chemotaxis for fibroblasts or endothelial cells; the preferred glycosaminoglycans are said to be heparin, heparan sulfate and alginate, although it should be noted that alginate is not in fact a glycosaminoglycan.

The infused dressings of the present invention may be prepared using commercially available dispersions of the individual components. The Multidex® described above is manufactured by DeRoyal Industries, Inc. Collagen type I from human placenta or bovine collagen is a commercial product diluted with sterile normal saline to the concentration levels noted above, e.g., 2 μg/cm². Commercial alpha-tocopherol acetate solution is added with good mixing to the diluted collagen suspension to achieve the desired level, e.g., 10 μg/ml. The resultant colloidal suspension had a pH of 4.5 to 5.5 to match that of the Maltodextrin. The composition is preferably added to the liquid phase absorptive collagen, hydrophilic foam, hydrocolloid, hydrogel, hydrogel sheets, polyacrylate or similar porous materials to create the dressing. If such is desired, standard medically acceptable gelling materials, e.g. cellulose, may be included in the compositions.

The prepared compositions preferably are maintained under refrigeration or maintained at room temperature. When kept under refrigeration, the suspensions will maintain their effectiveness for extended periods, i.e. months.

Although the compositions may be used as the aqueous colloidal suspension of Maltodextrin, collagen type I, and alpha-tocopherol, per se, it is also possible to add small amounts of an antibiotic, e.g. neomycin sulfate, normally used for topical applications. Such addition of a topical antibiotic is not necessary to promote wound healing by the present compositions. Such addition is solely as a matter of convenience in the general management of wounds.

The wounds were first thoroughly cleansed and decontaminated as per standard medical practice and any necrotic tissue was debrided to leave as clean and sterile a wound surface as possible. One of the dressings described infused with the saline-Maltodextrin-collagen type I-alpha-tocopherol is applied to cover the wound. The wound healing dressing may be reapplied daily to the wound surfaces. This procedure should be followed until new epithelial tissue completely closes the wound surface, at which time, application of the wound healing composition was discontinued.

While the foregoing representative dressings and details have been shown for the purpose of illustration and invention, it will be apparent to those skilled in the art that various changes and modifications may be made in the treatment of surface wound using a suspension of particles of collagen type I, ascorbic acid, and alpha-tocopherol with starch hydrolysate having a low DE of less than 35, which are chemotactic for fibroblasts and endothelial cells, dry suspensions, gel, aqueous suspensions, articles comprising combinations of the collagen, ascorbic acid, alpha-tocopherol with starch hydrolysate having a DE less than 35 as a suitable carrier selected from the group consisting of a gauze, sterile bandage or a tape therein without departing from the spirit or the scope of the invention. It is intended that all such changes and modifications will be embraced within this invention, provided they fall within the appended claims.

What is claimed in this invention is:

1. A method for promoting the healing of a surface wound, comprising the steps of:
applying to the wound surface a medical dressing infused with a suspension of starch hydrolysate having a low dextrose equivalent and ascorbic acid whereby the composition applied to the wound is chemotactic for fibroblasts or endothelial cells to effectively promote the healing process.

2. The method of claim 1 wherein two or more vitamins are combined in the suspension.

3. The method of claim 2 wherein the combined vitamins are vitamin C and vitamin E.

4. The method of claim 1 wherein the suspension includes collagen and alpha-tocopherol.

5. The method of claim 1 wherein the vitamin C is present in the suspension in concentrations in the order of about 0.5-20 weight percent of the blended composition.

6. The method of claim 4 wherein the concentration of vitamin E in the suspension is on the order of 8-10 μg/ml

7. The method of claim 1 wherein the starch hydrolysate in the suspension has a DE of less than about 35.

8. The method of claim 1 wherein the starch hydrolysate in the suspension has a DE between about 5 and about 25.

9. The method of claim 1 wherein the starch hydrolysate has a DE between about 9 and about 13.

10. A method of treating skin wounds which comprises the steps of infusing a carrier dressing with starch hydrolysate having a DE of less than about 50 and applying the carrier loosely to the wound.

11. The method of claim 10 wherein the starch hydrolysate has a descriptive ratio of about 2 or greater, the descriptive ratio being the quotient obtained by dividing the sum of the percentages of saccharide, dry basis, having a degree of polymerization of 1 to 6 by the DE.

12. The method of claim 1 wherein the suspension is gelled before infusion into the carrier dressing.

13. The method of claim 1 wherein collagen type I is added to the suspension in concentrations in the order of about 0.2 to 2 μg/cm².

14. The method of claim 1 including collagen type I in chemotactically effective amounts.

15. A method for promoting the healing of a surface wound, which method comprises, applying to the wound surface a medical dressing infused with a suspension of starch hydrolysate having a DE of less than 35 itself comprised of a mixture of: collagen type I; and alpha-tocopherol wherein the vitamin C is chemotactic for fibroblasts or endothelial cells and the collagen type I is from human placenta wherein the infused dressing is applied repeatedly to the wound surface during the healing to effectively promote the healing process.

16. A method for promoting the healing of a surface wound, which method comprises, applying to the wound surface a medical dressing infused with a suspension of starch hydrolysate having a DE of less than 35 itself comprised of an admixture of: collagen type I and vitamin C wherein the alpha-tocopherol is applied to a number of times to the wound surface during the healing to effectively enhance the healing process wherein the amounts of alpha-tocopherol in the suspension is below that which produces an inhibition of the migration of the cells into the wound area, and is below that which produces undesirable local inflammation.

17. The composition of claim 16 wherein two or more vitamins are present in the suspension in the pH range of 4.5 to 5.5.

18. The composition of claim 17 wherein the starch hydrolysate having a DE of less than 35 as the suspending component.

19. A composition for use in promoting the healing of surface wound, comprising a medical dressing infused with a suspension of starch hydrolysate with a DE of less than 35.

20. A medical dressing for promoting the healing of surface wound, comprising a pourous dressing infused with an aqueous suspension of vitamins C and E and collagen type I, in admixture with a starch hydrolysate that has a DE less than 35 that exhibits a chemotactic effect on fibroblasts or endothelial cells, wherein the collagen type I is purified from human placenta and the collagen is in the suspension at a concentration of 2 μg/cm².

21. A medical dressing comprising a dressing infused with a suspension of a major amount of collagen type I and vitamin E in admixture with a minor amount of ascorbic acid, and starch hydrolysate having a DE less 35 as the suspension.

22. The composition of claim 21 wherein the composition is chemotactic for fibroblasts or endothelial cells.

23. The composition of claim 22 wherein vitamin E is present at about 8-10 μg/ml.

24. A composition for use in promoting the healing of surface wound, comprising a medical dressing infused liquid aqueous suspension collagen type I present in the suspension in the order of 0.2-2 ug/cm²; the 1% ascorbic acid in the Matodextrin, NF as in admixture with a starch hydrolysate that has a DE less than 35 while alpha-tocopherol present in the order of 8-10 μg/ml in the suspension are agents that greatly accelerate the rate of wound healing and reduces scarring in this invention.
